(19) United States
(12) Patent Application Publication
(10) Pub. No.: US 2023/0240573 A1
(43) Pub. Date: Aug. 3, 2023

(54) ARTIFICIAL INTELLIGENCE PSYCHOLOGICAL STRESS DETECTION DEVICE

(71) Applicants: Cheng-Ta LI, Taipei City (TW); Shuo-Hong HUNG, Kaohsiung City (TW)

(72) Inventors: Cheng-Ta LI, Taipei City (TW); Shuo-Hong HUNG, Kaohsiung City (TW)

(21) Appl. No.: 17/586,899

(22) Filed: Jan. 28, 2022

(51) Int. Cl. A61B 5/16; G16H 40/63; A61B 5/00; A61B 5/0205; G01J 3/28

(52) U.S. Cl. CPC A61B 5/165; G16H 40/63; A61B 5/0022; A61B 5/7264; A61B 5/02055; G01J 3/28; A61B 2560/0242; A61B 5/024

(57) ABSTRACT

An artificial intelligence psychological stress detection device of the present invention includes a mental sensor chip, a communication module, and a microcontroller unit. The mental sensor chip is configured to sense a user's physiological characteristic(s) and/or behavior characteristic(s) and/or environmental characteristic(s). The communication module is configured to communicate with a mobile device. The microcontroller unit is connected to the mental sensor chip and the communication module, and configured to send the user's physiological characteristic(s) and/or the environmental characteristic(s) and/or the behavior characteristic(s) obtained from the mental sensor chip to the mobile device via the communication module.

### BACKGROUND OF THE INVENTION

### 1. Field of the Invention

The present invention relates to an artificial intelligence psychological stress detection device, which may be a portable or wearable device.

### 2. Description of Related Art

According to the report from the World Health Organization (WHO), there are about 540 million people in the world suffering from mental illness. Taking Mainland China as an example, there are up to 100 million psychiatric patients, but there are only 20 thousand psychiatrists.

Therefore, every psychiatrist bears an extremely heavy medical burden as he/she has to face 5 thousand patients in average.

Moreover, when a patient visits a hospital for psychotherapy, he/she has to fill in a long evaluation questionnaire. It takes the patient at least 30 minutes to fill in the questionnaire, which is time-consuming and laborious. Furthermore, the answers to the questionnaire tend to reflect merely the subjective feelings of the patient, and it lacks objective data for performing a quantitative analysis to give effective diagnosis and treatment to the mental illness. In addition, there is often an alienation between the patient and the psychiatrist, so that the patient cannot clearly and completely convey his/her condition to the psychiatrist. Besides, a face-to-face diagnosis takes only 10 minutes in average, and follow-up diagnoses are required only every two weeks, resulting in the ineffective discovery and track for mental illness.

Therefore, it is desirable to provide an improved psychological stress detection device to mitigate and/or obviate the aforementioned problems.

### SUMMARY OF THE INVENTION

In view of this, the present invention aims to provide an artificial intelligence psychological stress detection device, which can analyze a person's psychological stress, and give care and support to the person. In particular, the artificial intelligence psychological stress detection device of the present invention can realize the following functions:

The first function is the internet of things (IoT) sensing, involving various items such as heartbeat, respiration, intensity or spectrum of light, skin impedance, sleep, exercise, and so on; the sensing may further be combined with the global positioning system (GPS).

The second function is the artificial intelligence prediction, wherein the information collected by the IoT sensing and the information of the hospital's electronic medical record report are both inputted into the artificial intelligence (AI), for the AI to predict the user's mental conditions; the prediction can be used to assist a psychiatrist in diagnosis, and further assist the psychiatrist in choosing a medical solution for a patient, thereby realizing a precise treatment.

The third function is the artificial intelligence support, including giving regard and care by a chat robot; in particular, it is possible to introduce a kit of natural language processing (NLP) to qualitatively and quantitatively analyze the user's emotional expression, so as to interact more closely with the user.

Other objects, advantages, and novel features of the invention will become more apparent from the following detailed description when taken in conjunction with the accompanying drawings.

### BRIEF DESCRIPTION OF THE DRAWINGS

FIG. 1 shows a systemic architectural diagram of the environmental light information pressure detection system according to one embodiment of the present invention;

FIG. 2 shows a block diagram of the artificial intelligence psychological stress detection device according to one embodiment of the present invention;

FIG. 3 shows a sectional view of the light sensing structure of the environmental light sensor according to one embodiment of the present invention;

FIG. 4 shows the eighteen bands covered by the environmental light sensor according to one embodiment of the present invention;

FIG. 5 shows a working flowchart of the artificial intelligence psychological stress detection device according to one embodiment of the present invention;

FIG. 6 shows a working flowchart of the mobile application of the mobile device according to one embodiment of the present invention;

FIG. 7 shows relationships between different subjects with high or low physical and psychological stresses with respect to irradiance according to the test of the present invention;

FIG. 8 shows an architectural diagram of the artificial intelligence psychological stress detection device according to one embodiment of the present invention;

FIG. 9 shows the receiver operating characteristic curves for various machine learnings;

FIG. 10 shows a schematic diagram of the medical-grade artificial intelligence behavior characteristic(s) algorithm according to one embodiment of the present invention;

FIG. 11 shows a schematic diagram of the personalized timely care algorithm according to one embodiment of the present invention; and

FIG. 12 shows a schematic diagram of the probabilistic prediction algorithm for user behavior pattern according to one embodiment of the present invention.

### DETAILED DESCRIPTION OF THE EMBODIMENT

Different embodiments of the present invention are provided in the following description. These embodiments are meant to explain the technical content of the present invention, but not meant to limit the scope of the present invention. A feature described in an embodiment may be applied to other embodiments by suitable modification, substitution, combination, or separation.

It should be noted that, in the present specification, when a component is described to have an element, it means that the component may have one or more of the elements, and it does not mean that the component has only one of the element, except otherwise specified.

Moreover, in the present specification, the ordinal numbers, such as “first” or “second”, are used to distinguish a plurality of elements having the same name, and it does not mean that there is essentially a level, a rank, an executing order, or an manufacturing order among the elements, except otherwise specified. A “first” element and a “second” element may exist together in the same component, or alternatively, they may exist in different components, respectively. The existence of an element described by a greater ordinal number does not essentially mean the existent of another element described by a smaller ordinal number.

In the present specification, a description “a feature X ‘or ’ or ‘and/or’ another feature Y” means that X exists alone, Y exists alone, or X and Y exist together; a description “a feature X ‘and’ another feature Y means that X and Y exist together; the terms “include”, “contain”, “have”, or “comprise” means “include but not limited thereto”, except otherwise specified.

Moreover, in the present specification, the terms, such as “top”, “bottom”, “left”, “right”, “front”, “back”, or “middle”, as well as the terms, such as “on”, “above”, “under”, “below”, or “between”, are used to describe the relative positions among a plurality of elements, and the described relative positions may be interpreted to include their translation, rotation, or reflection.

Moreover, in the present specification, when an element is described to be arranged “on” another element, it does not essentially mean that the elements contact the other element, except otherwise specified. Such interpretation is applied to other cases similar to the case of “on”.

Moreover, in the present specification, the terms, such as “preferably” or “advantageously”, are used to describe an optional or additional element or feature, and in other words, the element or the feature is not an essential element, and may be ignored in some embodiments.

Moreover, in the present specification, when an element is described to be “suitable for” or “adapted to” another element, the other element is an example or a reference helpful in imagination of properties or applications of the element, and the other element is not to be considered to form a part of a claimed subject matter; similarly, except otherwise specified; similarly, in the present specification, when an element is described to be “suitable for” or “adapted to” a configuration or an action, the description is made to focus on properties or applications of the element, and it does not essentially mean that the configuration has been set or the action has been performed, except otherwise specified.

Moreover, each component may be realized as a single circuit or an integrated circuit in suitable ways, and may include one or more active elements, such as transistors or logic gates, or one or more passive elements, such as resistors, capacitors, or inductors, but not limited thereto. Each component may be connected to each other in suitable ways, for example, by using one or more traces to form series connection or parallel connection, especially to satisfy the requirements of input terminal and output terminal. Furthermore, each component may allow transmitting or receiving input signals or output signals in sequence or in parallel. The aforementioned configurations may be realized depending on practical applications.

Moreover, in the present specification, the terms “system”, “apparatus”, “device”, “module”, or “unit”, and so on, refer to an electronic element, or a digital circuit, an analogous circuit, or other general circuit, composed of a plurality of electronic elements, and there is not essentially a level or a rank among the aforementioned terms, except otherwise specified.

Moreover, in the present specification, two elements may be electrically connected to each other directly or indirectly, except otherwise specified. In an indirect connection, one or more elements, such as resistors, capacitors, or inductors may exist between the two elements. The electrical connection is used to send one or more signals, such as DC or AC currents or voltages, depending on practical applications.

Moreover, a terminal or a server may include the aforementioned element(s), or be implemented in the aforementioned manner(s).

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

(Artificial Intelligence Psychological Stress Detection Device with Respect to Environmental Light Information)

Researches have reported that light can influence a person's mood and psychological stress. When a person is exposed to untimely or barely changing light, the person's biological rhythm can be asynchronous with the person's behavior rhythm. Moreover, the circadian regulation also permeates many physiological systems, including hypothalamic-pituitary-adrenal (HPA) axis and neurotransmitter such as melatonin. In particular, the activation of HPA axis is related to a person's reaction when he/she is adapting to external environment or coping with life stress, and the secretion of melatonin is related to a person's sleep regulation, mood, or behavior.

It is known from prior researches that irregular light period may lead to mental illness. However, the prior researches have not discussed about different effects of different bands of light on human body. The prior researches cannot track the complete environmental light information for a long time in real life mainly because there lacks a suitable light sensor. Known optical instruments include spectrometer, illuminometer, and direct illumination. The spectrometer is disadvantageous for its heaviness, high power consumption, and inapplicability to become a portable or wearable device. The illuminometer is disadvantageous for it can lose the light information of different wavelengths, and it can hardly stand by for more than one day. The direct lighting is disadvantageous for it can only be used to roughly observe the effect given by a specific type of light bulb on human body, but it is difficult to analyze the respective effects given by different bands of light, as well as it is limited to a fixed field so that it cannot be widely used in real life.

For this purpose, the present invention provides an artificial intelligence psychological stress detection device, which includes a miniaturized environmental light sensor module, and it is portable or wearable in aspect of its volume; it can trace the complete light information from violet light to infrared light by its multiple channels in aspect of its function; it can continuously record the light information for a long time because of its low power consumption in term of its performance.

In one circumstance, the present invention can further be combined with big data researches. According to the present invention, it is also verified that machine learning can be utilized to deal with the environmental light analysis; in particular, the Support Vector Machine (SVM) model can be used to identify a field where it locates, with an accuracy up to 95%.

In another circumstance, according to the present invention, it is possible to discuss the relationship between light and psychological stress. The results show that the people who self-assess themselves having high psychological stress generally lack exposure to green light, red light, and infrared light.

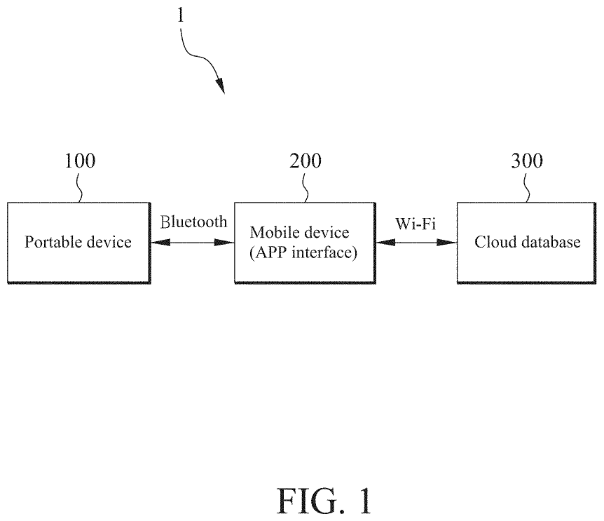

FIG. 1 shows a systemic architectural diagram of the environmental light information pressure detection system 2 according to one embodiment of the present invention. As shown in FIG. 1, the environmental light information pressure detection system 1 of the present invention includes an artificial intelligence psychological stress detection device 100, a mobile device 200, and a cloud server 300 including a cloud database. The artificial intelligence psychological stress detection device 100 may be a wearable device. The artificial intelligence psychological stress detection device 100 may be communicated with the mobile device 200 via Bluetooth, but not limited thereto. In some embodiments, the artificial intelligence psychological stress detection device 100 may be integrated with the mobile device 200. In other words, the chip of the artificial intelligence psychological stress detection device 100 may be implanted into the mobile device 200. The mobile device 200 may be communicated with the cloud server 300 via networks such as Wi-Fi, 3G, 4G, 5G, and so on, but not limited thereto.

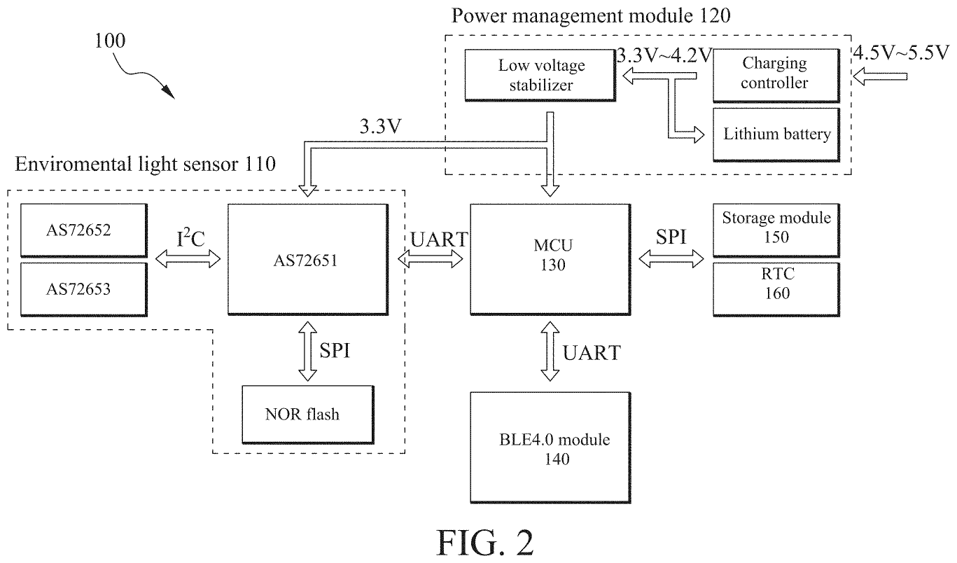

FIG. 2 shows a block diagram of the artificial intelligence psychological stress detection device 100 according to one embodiment of the present invention. As shown in FIG. 2, the artificial intelligence psychological stress detection device 100 of the present invention includes an environmental light sensor 110, a power management module 120, a microcontroller unit (MCU) 130, a communication module (BLE4.0 module) 140, a storage module 150, and a real-time clock (RTC) 160.

The environmental light sensor 110 is used to collect environmental light information.

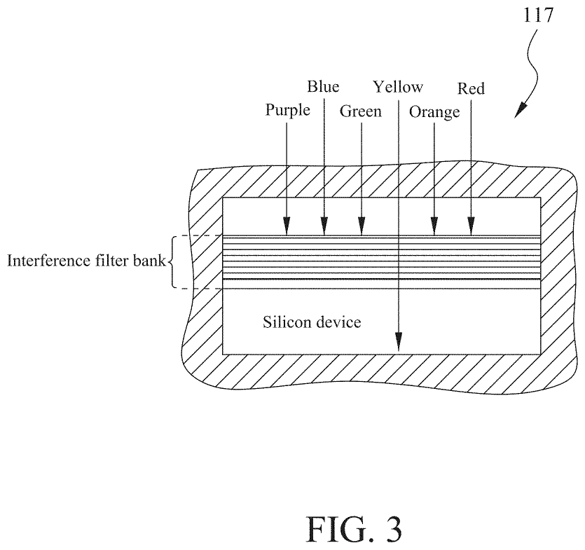

FIG. 3 shows a sectional view of the light sensing structure 117 of the environmental light sensor 110 according to one embodiment of the present invention, wherein an interference filter bank (composed of a plurality of interference filter sheets arranged in order) is directly deposited on a silicon component, so the environmental light sensor 110 can have a miniaturized volume, and can save power.

Referring back to FIG. 2, the environmental light sensor 110 is composed of three chips, AS72651, AS72652, and AS72653. Each chip can be used to collect (or receive) light information of six bands, so the environmental light sensor 110 can cover the light information of totally eighteen bands from violet light to infrared light.

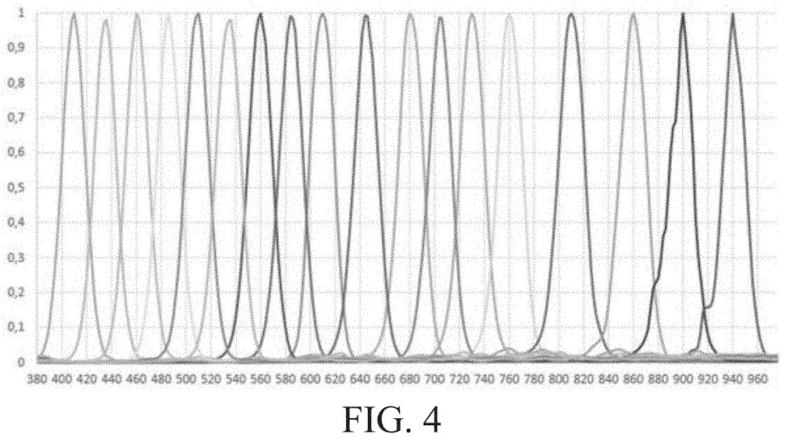

FIG. 4 shows the eighteen bands covered by the environmental light sensor 110 according to one embodiment of the present invention, wherein each band is indicated representatively by its peak wavelength, but it may alternatively be indicated representatively by its center wavelength. The following description follows the same definition.

Referring back to FIG. 2, the power management module 120 is used to manage a battery to supply power to or to charge power from other connected modules or detailed components.

The MCU 130 is used to control the communication among modules or detailed components, wherein when designing the MCU 130, it should be noted the following guidelines of the present invention:

The first guideline is that, since there are eighteen bands of light information needed to be collected, it correspondingly requires a serial cache having at least 128 bytes. Without such serial cache, a part of information may possibly be lost during transmission.

The second guideline is that, the electrical voltage should be controlled to be a low voltage of 3.3V, so as to meet the requirement of saving power.

The third guideline is that, although there already exist a kind of firmware providing 5V voltage with corresponding 16 Mhz oscillator and another kind of firmware providing 3.3V voltage with corresponding 8 Mhz oscillator, but the 5V voltage provided by the former one does not satisfy the aforementioned second guideline, and the 8 MHz oscillator provided by the latter one is too slow and will result in abnormal communication between the MCU 130 and the environmental light sensor 110. Therefore, the MCU 130 should be overclocked up to, for example, 12 MHz.

The communication module 140 is used for wireless transmission, and it may be a Bluetooth module, but not limited thereto.

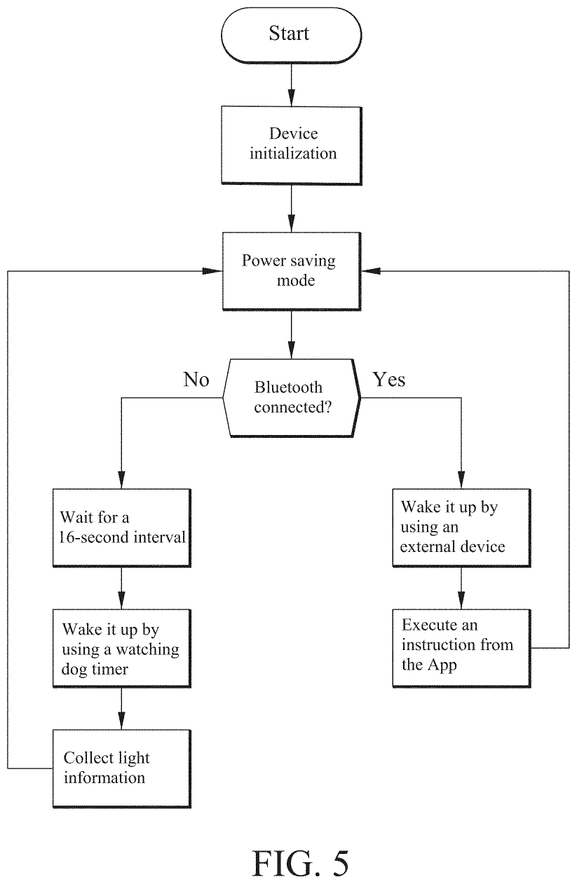

FIG. 5 shows a working flowchart of the artificial intelligence psychological stress detection device 100 according to one embodiment of the present invention, wherein the artificial intelligence psychological stress detection device 100 of the present invention enters different working modes depending on whether the communication module 140 (for example, the Bluetooth in this case) is connected to the mobile device 200. If the Bluetooth is disconnected, then the device 100 is set to receive the environmental light information once every sixteen seconds (but it is still possible to use other time intervals); if the Bluetooth is connected, then the device 100 executes the instruction received from the mobile device 200, associated with, for example, data upload or timing synchronization. The detailed steps may be referred to the remarks in the flowchart of FIG. 5.

Referring back to FIG. 2, the RTC 160 is used to record the real time. In particular, the timestamp of the real-time clock may be combined with the user's physiological characteristic(s) and/or behavior characteristic(s) and/or environmental characteristic(s), so as to forming rhythm data. When there is a great difference (for example, a great delay) between a proposed rhythm data (for example, a biological rhythm from a healthy person) and the user's rhythm data obtained by the detection, a notification may be issued to notify the user to adjust his/her daily routine.

The storage module 150 is used to temporarily or permanently store the aforementioned environmental light information and time information, and it may be a microSD card for example, but not limited thereto.

As can be seen in Table 1 below, when comparing the present invention with the existing instruments on the market, the artificial intelligence psychological stress detection device 100 of the present invention has a volume smaller than ⅙ of the volume of the existing instruments, and has a weight lighter than 1/9 of the weight of the existing instruments, so it can realize the portable or wearable requirements. Besides, the present invention uses a lithium battery of 500 mAh, which can continuously collect the environmental light information for more than 37 hours.

TABLE 1

| | | | | The present |
|---|---|---|---|---|
| Apparatus | Spectrometer | illuminometer | Spectrometer | invention |
| Model number | SMUSB | LX-1128SD | HR-450 | The present |
| | | | | invention |
| Volume | 207.5 cm³ | 541.6 cm³ | 405.6 cm³ | 32.5 cm³ |
| Covered bands | 305 nm~1050 nm | None | 380 nm~780 nm | 410 nm~940 nm |
| Light information | Multichannel | White light | Multichannel | Multichannel |
| type | | | | |
| Weight | 263 g | 489 g | 276 g | 28 g |
| Stand-by time | None | None | More than 5 | More than 37 |
| | | | hours | hours |
| Wearable device | No | No | No | Yes |

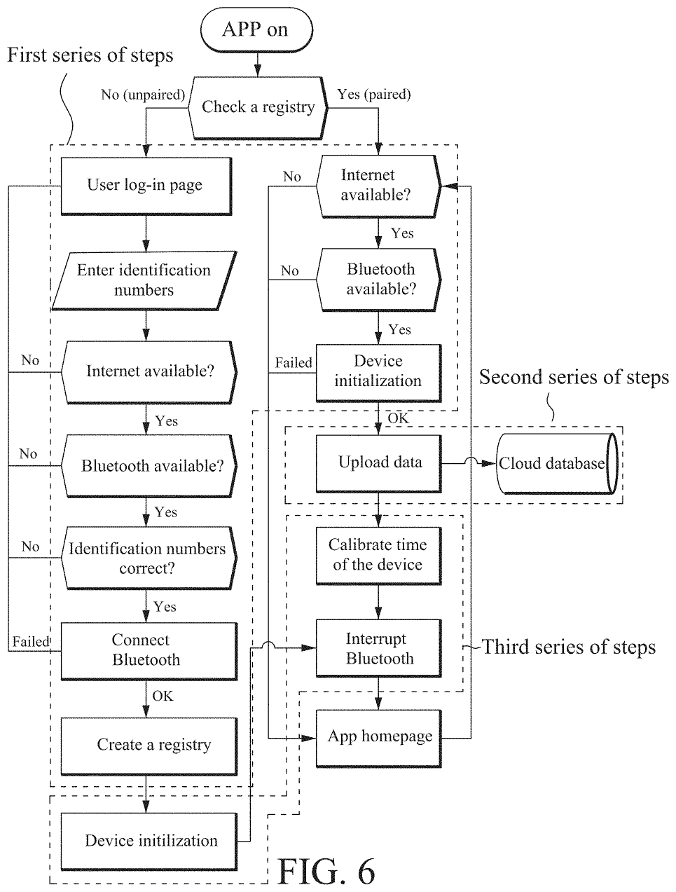

FIG. 6 shows a working flowchart of mobile application (APP) mobile device 200 according to one embodiment of the present invention, which can be divided into three main series of steps:

The first series of steps are to confirm whether the network of mobile device 200 (for example, the Bluetooth function) has been enabled, as well to verify whether the user's identity is correct.

The second series of steps are to upload the environmental light information collected by the artificial intelligence psychological stress detection device 100 via the mobile device 200 to a cloud database on a dedicated server 300.

The third series of steps are to issue an instruction via the mobile device 200, in order to initialize the artificial intelligence psychological stress detection device 100 and to synchronize its time with the mobile device 200's time.

The detailed steps can be directly referred to the description in the flowchart of FIG. 6.

(Machine Learning)

The artificial intelligence psychological stress detection device 100 of the present invention was used in seven different fields, and it randomly moved among seven fields and collected light information therefrom. It collected at least 3,000 data of environmental light information in each field, so it totally collected at least 21,000 data of environmental light information. The collected environmental light information in each field is allocated as training group data, validation group data, and test group data in a ratio of 4:1:1. In particular, the present invention chose the seven fields in the building of Psychiatry Department of Taipei Veterans General Hospital, wherein Field 1 is the elevator, Field 2 is the first floor, Field 3 is the corridor on the third floor, Field 4 is the elevator room on the third floor, Field 5 is the protective isolation ward, Field 6 is the stair, and Field 7 is the general ward, for example, but not limited thereto.

Next, the present invention introduces two different kinds of machine learning modes, the SVM and the neural network to recognize these fields. A machine learning model is typically constructed on the cloud server 300, but it may also be constructed in the mobile device 200 itself if the mobile device 200 has enough hardware capability.

The results show that the SVM working with the Radial Basis Function (RBF) kernel can achieve a test accuracy up to 95%. The distribution of actual fields and predicted fields by the SVM are shown in Table 2-1 below.

In comparison, the neural network using the basic three-layer architecture neural network can achieve a test accuracy of 79%. The distribution of the actual fields and predicted fields of the neural network are shown in Table 2-2 below.

TABLE 2-1

| | Predicted | Predicted | Predicted | Predicted | Predicted | Predicted | Predicted |
|---|---|---|---|---|---|---|---|
| | field 0 | field 1 | field 2 | field 3 | field 4 | field 5 | field 6 |
| Actual | 99% | 0.8% | 0% | 0.2% | 0% | 0% | 0% |
| field 0 | | | | | | | |
| Actual | 0% | 86.4% | 3.6% | 6.4% | 0% | 3.2% | 0.4% |
| field 1 | | | | | | | |
| Actual | 0% | 2% | 96.6% | 0.4% | 0% | 0.2% | 0.8% |
| field 2 | | | | | | | |
| Actual | 0% | 8.8% | 0.02% | 88% | 0% | 0.8% | 0.4% |
| field 3 | | | | | | | |
| Actual | 0% | 0.2% | 0% | 0% | 99.4 % | 0.4% | 0% |
| field 4 | | | | | | | |
| Actual | 0% | 2% | 2.4% | 0.1% | 0% | 94.4% | 0.2% |
| field 5 | | | | | | | |
| Actual | 0% | 0.6% | 0.6% | 0.4% | 0% | 0.6% | 97.8% |
| field 6 | | | | | | | |

TABLE 2-2

| | Predicted | Predicted | Predicted | Predicted | Predicted | Predicted | Predicted |
|---|---|---|---|---|---|---|---|
| | field 0 | field 1 | field 2 | field 3 | field 4 | field 5 | field 6 |
| Actual | 99% | 0.5% | 0% | 0.2% | 0% | 0% | 0% |
| field 0 | | | | | | | |
| Actual | 0.2% | 61.2% | 7.1% | 17.3% | 0.2% | 5.1% | 8.6% |
| field 1 | | | | | | | |
| Actual | 0.09% | 3.9% | 73% | 8.6% | 0.3% | 4.6% | 9.5% |
| field 2 | | | | | | | |
| Actual | 0.09% | 15% | 7.7% | 67% | 0% | 4.8% | 5.2% |
| field 3 | | | | | | | |
| Actual | 0% | 0.2% | 0.5% | 0% | 99% | 0% | 0% |
| field 4 | | | | | | | |
| Actual | 0% | 3.8% | 5.7% | 13.6% | 0.1% | 72% | 4.8% |
| field 5 | | | | | | | |
| Actual | 0% | 1.7% | 5% | 0.8% | 0% | 0.3% | 90% |
| field 6 | | | | | | | |

(Relationship Between Environmental Light Information and Stress)

The present invention compares the environmental light information with the psychological stress data, trying to obtain the correlation between them. The stress data may come from questionnaires, physiological characteristic(s), or behavior characteristic(s). The questionnaires may include a physical and psychological stresses scale or a life stress scale. The physical and psychological stresses scale is used to assess the psychological pressure felt by the subjects themselves, while the life stress scale is used to assess the pressure brought by the environment to the subjects.

In the test of the present invention, the subject wears the artificial intelligence psychological stress detection device 100 of the present invention during the working day, continuously recording the environmental light information for at least half a day. Finally, the Mann-Whitney U test is used to perform statistical analysis.

The recorded environmental light information may be presented in various visualized forms, for example:

The first form is a spectrogram, which can be inputted into a graphical recognition architecture such as the Convolutional Neural Network (CNN) for training and prediction, and may be combined with big data researches.

The second form is a broken line graph, which can show the respective bands' changes with respect to time following the subject's movement in his/her diary life fields.

The third form is a box diagram, which can show the distribution and trend of the illumination on the subjects.

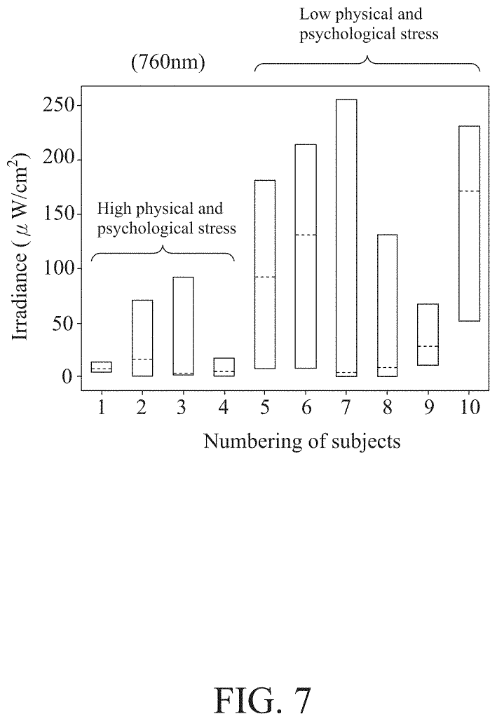

FIG. 7 shows relationships between different subjects with high or low physical and psychological stresses with respect to irradiance according to the test of the present invention. The test results in FIG. 7 show that people with high physical and psychological stresses receive less light in their lives.

Moreover, the irradiances (unit: μW/cm²) of the eighteen bands of lights collected by the environmental light sensors 110 of the artificial intelligence psychological stress detection devices 100 of the present invention worn on the users are shown in Table 3, Table 4, and Table 5.

Based on these data, a further analysis on the light bands received by the people with high physical and psychological stresses shows that they mainly lack green light (including the light of wavelength of 510 nm and 535 nm), red light (including the light of wavelength of 680 nm, 705 nm, and 730 nm), and infrared light (including the light of wavelength of 760 nm, 810 nm, and 900 nm), totally eight bands of light.

TABLE 3

| | 410 nm | 435 nm | 460 nm | 485 nm | 510 nm | 535 nm |
|---|---|---|---|---|---|---|
| 1 | 24.29 | 56.53 | 47.33 | 40.78 | 36.22 | 42.43 |
| 2 | 110.17 | 192.64 | 78.48 | 322.35 | 114.84 | 126.8 |
| 3 | 125.38 | 198.15 | 150.25 | 283.34 | 139.69 | 173.74 |
| 4 | 213.01 | 515.65 | 342.07 | 578.73 | 255.28 | 298.03 |
| 5 | 203.37 | 1290.46 | 1093.44 | 374.51 | 414.8 | 1728.91 |
| 6 | 41.38 | 214.26 | 235.19 | 103.48 | 287.18 | 565.51 |
| 7 | 166.06 | 1007.15 | 1018.77 | 375.2 | 444.27 | 1500.86 |
| 8 | 16.88 | 20.66 | 33.41 | 31.46 | 36.68 | 52.87 |
| 9 | 298.52 | 491.84 | 296.82 | 565.29 | 357.31 | 334 |
| 10 | 326.13 | 2031.52 | 1269.49 | 1796.42 | 861.24 | 4114.13 |

TABLE 4

| | 560 nm | 585 nm | 610 nm | 645 nm | 680 nm | 705 nm |
|---|---|---|---|---|---|---|
| 1 | 76.37 | 32.82 | 57.3 | 40.92 | 17.49 | 19.14 |
| 2 | 137.78 | 262.74 | 209.71 | 249.06 | 39.27 | 51.41 |
| 3 | 160.11 | 239.64 | 263.29 | 231.37 | 97.74 | 104.47 |
| 4 | 484.42 | 477.08 | 637.34 | 407.21 | 34.2 | 63.18 |
| 5 | 412.68 | 513.55 | 4828.6 | 411.64 | 125.44 | 243.97 |
| 6 | 441.71 | 743.98 | 697.49 | 506.62 | 310.83 | 310.15 |
| 7 | 328.1 | 315.99 | 1915.38 | 324.78 | 88.54 | 186.36 |
| 8 | 63.17 | 60.22 | 57.74 | 56.99 | 59.58 | 59.86 |
| 9 | 366.9 | 493.5 | 408.08 | 519.05 | 212.82 | 220.02 |
| 10 | 3286.32 | 3096.16 | 8098.05 | 1106.14 | 348.47 | 804.62 |

TABLE 5

| | 730 nm | 760 nm | 810 nm | 860 nm | 900 nm | 940 nm |
|---|---|---|---|---|---|---|
| 1 | 16.06 | 16.23 | 18.15 | 13.26 | 8.88 | 4.73 |
| 2 | 26.77 | 37 | 52.24 | 52.17 | 29.81 | 12.94 |
| 3 | 63.8 | 84.51 | 87.53 | 88.3 | 49.21 | 21.05 |
| 4 | 25.88 | 21.93 | 68.9 | 27.33 | 22.82 | 17.29 |
| 5 | 221.14 | 112.28 | 207.89 | 72.66 | 81.55 | 47.24 |
| 6 | 148.56 | 128.76 | 114.33 | 76.69 | 41.1 | 20.25 |
| 7 | 99.72 | 106.27 | 175.24 | 55.2 | 60.27 | 28.02 |
| 8 | 43.87 | 60.26 | 54.76 | 50.87 | 26.41 | 7.85 |
| 9 | 168.27 | 209.31 | 223.13 | 166.79 | 105.15 | 51.88 |
| 10 | 311.94 | 162.91 | 384.93 | 174.95 | 142.31 | 71.45 |

It is known that the conventional indoor fluorescent lamps lack the aforementioned kinds of green light, red light, and infrared light, but people with high physical and psychological stresses also spend most of their working days in the indoor spaces illuminated by fluorescent lamps. It is noted that, melancholia or depression can make people having a negative attitude and become less active, so that they go out less often and as a result cannot get specific light that is helpful to their health. According to the aforementioned test in the present invention, if people cannot get specific kinds of light helpful to their health, their physiologies and psychologies will be negatively affected, they will become even melancholy or depressed, and accordingly cause a vicious circle.

Therefore, when a user is found locating at a field where the specific spectral irradiances of green light, red light, and/or infrared light are lower than threshold values, the artificial intelligence psychological stress detection device 100 of the present invention may issue a notification to notify the user that he/she is advised to go out to expose to real sun light. The notification can be presented to any connected device in the form of sound or video.

(General Artificial Intelligence Psychological Stress Detection Device)

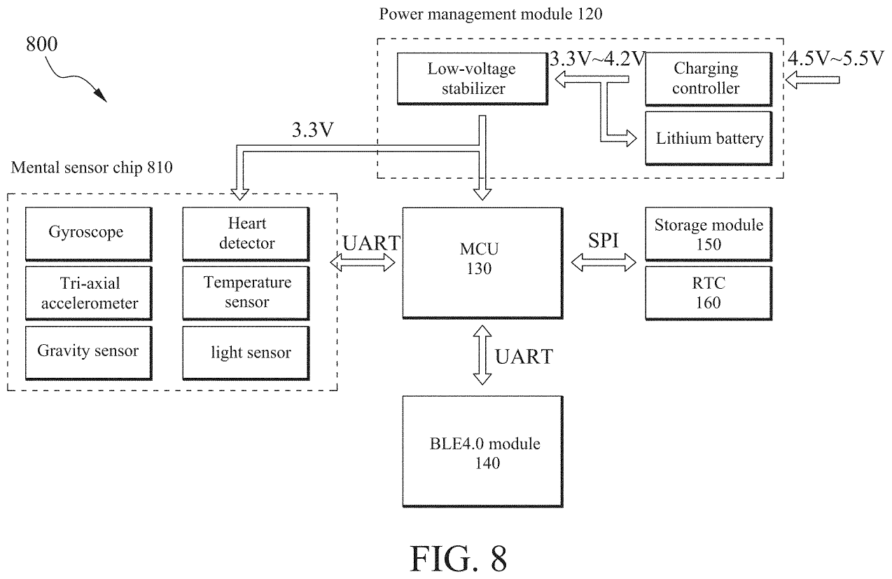

FIG. 8 shows an architectural diagram of the artificial intelligence psychological stress detection device 800 according to one embodiment of the present invention.

The artificial intelligence psychological stress detection device 800 of the present invention may be implemented by expanding the artificial intelligence psychological stress detection device 100 in the embodiment relevant to FIG. 2, with reference to FIG. 2 and FIG. 8 together. The artificial intelligence psychological stress detection device 800 may further include a mental sensor chip 810 in addition to the power management module 120, the MCU 130, the communication module 140, the storage module 150, and the RTC 160 of the device 100 of FIG. 3.

The mental sensor chip 810 may include various sensors, such as a gyroscope, a tri-axial accelerometer, a gravity sensor, a heart rate sensor, a temperature sensor, and a light sensor, to sense (or collect) the user's physiological characteristic(s) and/or behavior characteristic(s) and/or environmental characteristic(s), for example, heartbeat, respiration, intensity or spectrum of light illumination, skin impedance, sleep, activity, and so on. In particular, the light sensor may be an environmental light sensor 110 in FIG. 2.

The communication module 140 may perform data exchange by the Bluetooth Low Energy (BLE) technology working with the Advanced Encryption Standard (AES) 256-bit, so as to realize data transmission between the mobile device 200 and the artificial intelligence psychological stress detection device 800. The data may include the user's physiological characteristic(s) and/or behavior characteristic(s) and/or environmental characteristic(s). The mobile device 200 may send the data further to the cloud server 300, so that the data may be analyzed by the artificial intelligence on the cloud server 300.

(Artificial Intelligence)

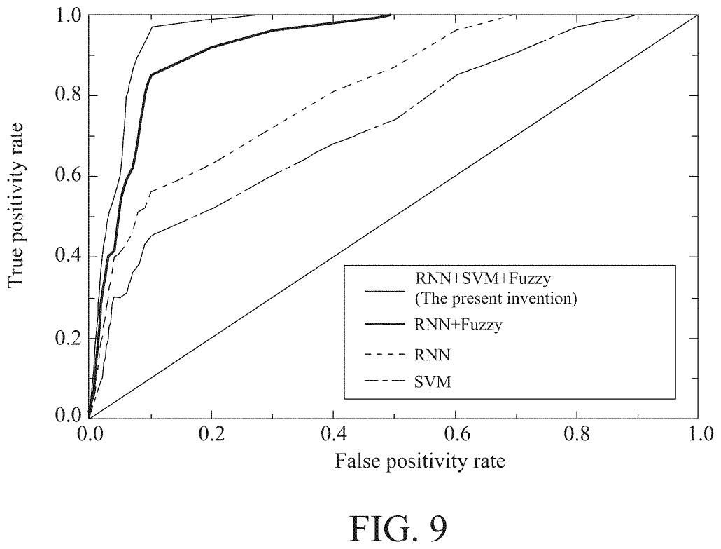

FIG. 9 shows the receiver operating characteristic curves (ROCs) for various machine learnings. The present invention may be combined with a recurrent neural network (RNN), a SVM, and a fuzzy neural network (FNN) to form an adaptive neuro-fuzzy inference system, which has extremely high prediction accuracy and extremely fast computation speed, particularly suitable for psychological stress detection.

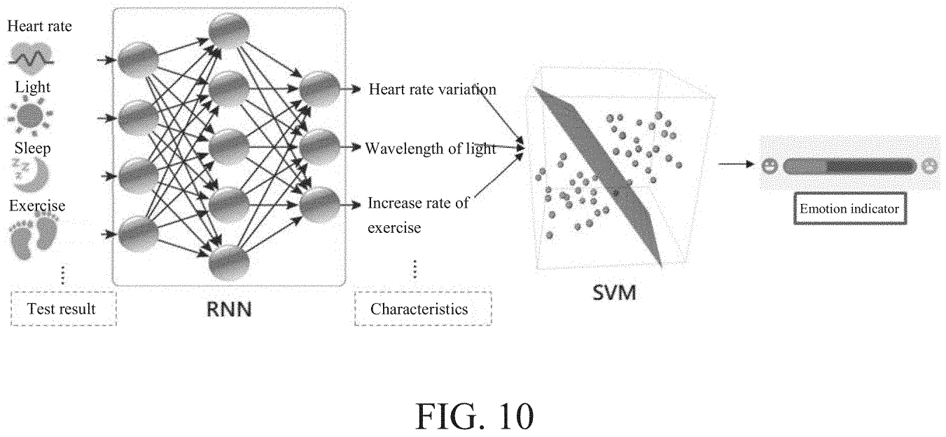

FIG. 10 shows a schematic diagram of the medical-grade artificial intelligence behavior characteristic(s) algorithm according to one embodiment of the present invention.

At the first stage, the algorithm of the present invention extracts personalized characteristics from the collected detection results (such as heartbeat, respiration, intensity or spectrum of light, skin impedance, sleep, exercise) by the RNN, and reclassifies them by the SVM, thereby effectively improving prediction accuracy and computation efficiency. The prediction result may be converted into a “mood index” or a “pressure index” that quantifies the user's mental state.

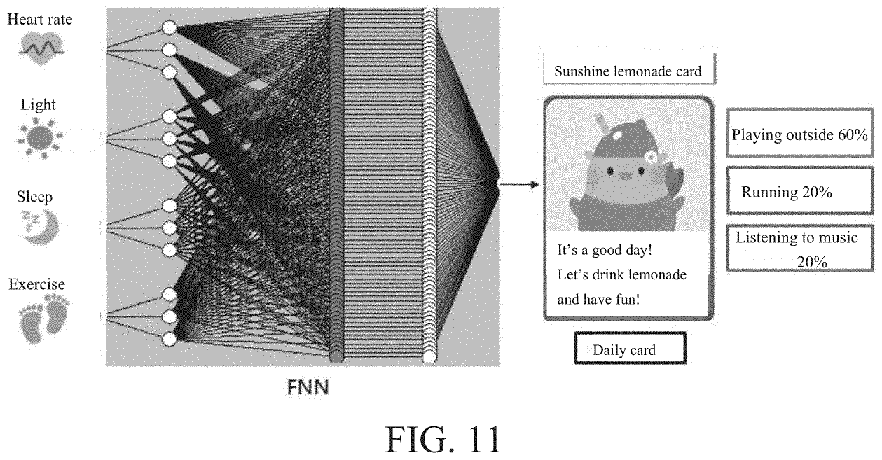

FIG. 11 shows a schematic diagram of the personalized timely care algorithm according to one embodiment of the present invention.

Following the previous description, at the next stage, the computation results are then represented by fuzzy decision making, different care solutions for melancholia are provide to the user, and the user may make the best choice for himself/herself among the presented care solutions. As shown in FIG. 11, after the detention results are inputted into the FNN, a care solution may be derived. The care solution may suggest that the user needs to arrange his/her today schedule with activities such as playing outside, running, listening to music, and so on, and the user is advised to spend time to do each activity until a proposed proportion of today's time, according to the FNN's fuzzy decision making result. In addition, the aforementioned care solutions may be presented in the form of “daily cards” with cute images or photos that brings healing effects to patients.

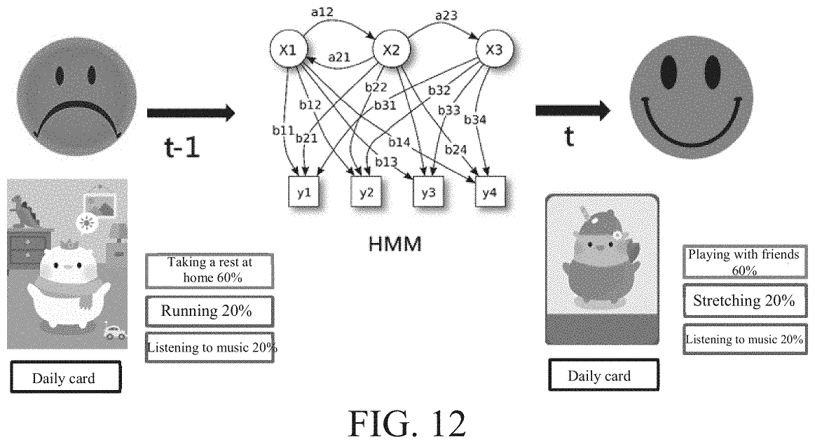

FIG. 12 shows a schematic diagram of the probabilistic prediction algorithm for user behavior pattern according to one embodiment of the present invention.

Following the previous description, at the final stage, the present invention uses the Hidden Markov Model (HMM) algorithm to perform probabilistic prediction for user's behavior patterns for a long time, keeping eye on the future changes in the user's depressive behavior, in order to provide preventive care that can avoid the user's mental condition to deteriorate to a serious condition that may cause his/her suicide. In the example of FIG. 12, at the present time point (t-1), the machine learning gives an advice that what the user is advised to do is taking a rest at home, running, and listening to music; while based on the probabilistic prediction via HMM, at the next time point (t), the machine learning gives an advice that what the user is advised to do is playing with friends, stretching, and listening to music.

Although the present invention has been explained in relation to its embodiments, it is to be understood that many other possible modifications and variations can be made without departing from the spirit and scope of the invention as hereinafter claimed.

What is claimed is:

1. An artificial intelligence psychological stress detection device, comprising:
a mental sensor chip, configured to sense a user's physiological characteristic(s) and/or behavior characteristic(s) and/or environmental characteristic(s);
a communication module, configured to communicate with a mobile device; and
a microcontroller unit, connected to the mental sensor chip and the communication module, and configured to send the user's physiological characteristic(s) and/or behavior characteristic(s) and/or environmental characteristic(s) obtained from the mental sensor chip to the mobile device via the communication module.

2. The artificial intelligence psychological stress detection device of claim 1, wherein the user's physiological characteristic(s) and/or behavior characteristic(s) and/or environmental characteristic(s) include heartbeat, respiration, intensity or spectrum of light illumination, skin impedance, sleep, or activity.

3. The artificial intelligence psychological stress detection device of claim 1, wherein the mental sensor chip includes a gyroscope, an accelerometer, a gravity sensor, a heart rate sensor, a temperature sensor, and/or a light sensor.

4. The artificial intelligence psychological stress detection device of claim 3, wherein the light sensor is an environmental light sensor, which includes an interference filter bank deposited on a silicon component.

5. The artificial intelligence psychological stress detection device of claim 4, wherein the environmental light sensor is configured to collect a plurality bands of light covering from violet light to infrared light.

6. The artificial intelligence psychological stress detection device of claim 5, wherein the bands of the light cover wavelengths from 410 nm to 940 nm.

7. The artificial intelligence psychological stress detection device of claim 5, wherein the bands of the light collected by the environmental light sensor are mapped to a prediction result by a machine learning module, and the prediction result is associated with a physical field where the artificial intelligence psychological stress detection device locates.

8. The artificial intelligence psychological stress detection device of claim 7, wherein the machine learning module is constructed on the mobile device itself, or the machine learning module is constructed on a cloud server communicating with the mobile device.

9. The artificial intelligence psychological stress detection device of claim 7, wherein the machine learning module is formed by a Recurrent Neural Network (RNN), a Support Vector Machine (SVM), a Deep Neural Network (DNN), and/or a Fuzzy Neural Network (FNN).

10. The artificial intelligence psychological stress detection device of claim 9, wherein the RNN extracts personalized characteristics from the user's physiological characteristic(s) and/or behavior characteristic(s) and/or environmental characteristic(s), and the SVM reclassifies the personalized characteristics.

11. The artificial intelligence psychological stress detection device of claim 9, wherein the FNN is used to derive a care solution from several possible solutions after detection results are inputted into the FNN.

12. The artificial intelligence psychological stress detection device of claim 9, wherein the FNN is configured to utilize fuzzy decision making.

13. The artificial intelligence psychological stress detection device of claim 1, wherein the microcontroller unit is configured to issue a notification when light of a specific wavelength obtained by an environmental light sensor of the mental sensor chip is lower than a spectral irradiance threshold value.

14. The artificial intelligence psychological stress detection device of claim 1, further comprising a real-time clock, connected to the microcontroller unit; wherein the microcontroller unit is configured to combine the user's physiological characteristic(s) and/or behavior characteristic(s) and/or environmental characteristic(s) with a timestamp of the real-time clock, and form rhythm data.

15. The artificial intelligence psychological stress detection device of claim 1, wherein the artificial intelligence psychological stress detection device is configured to determine a difference between a proposed rhythm data and the user's rhythm data.

16. The artificial intelligence psychological stress detection device of claim 1, wherein the artificial intelligence psychological stress detection device is configured to read a pressure index and issue a notification, and the notification includes a personalized adjustment strategy when the pressure index exceeds a threshold value.

17. The artificial intelligence psychological stress detection device of claim 1, wherein the artificial intelligence psychological stress detection device is a portable device or a wearable device.

18. The artificial intelligence psychological stress detection device of claim 1, wherein the artificial intelligence psychological stress detection device has a volume less than 32.5 cm³, a weight less than 28 g, and/or a stand-by time more than 37 hours.
